(19) United States
(12) Patent Application Publication
(10) Pub. No.: US 2016/0137450 A1
(43) Pub. Date: May 19, 2016

(54) ADHESIVE CONTAMINATION RESISTANT WEB PROCESSING UNIT

(71) Applicant: Curt G. Joa, Inc., Sheboygan Falls, WI (US)

(72) Inventors: MICHAEL T. HAHN, Plymouth, WI (US); James K. Melis, Glenbeulah, WI (US); Clinton H. Ylitalo, New Holstein, WI (US); Sudeep Ingole, Sheboygan, WI (US)

(21) Appl. No.: 14/942,605

(22) Filed: Nov. 16, 2015

Related U.S. Application Data
(60) Provisional application No. 62/080,613, filed on Nov. 17, 2014.

(51) Int. Cl. B65H 23/26; A61F 13/15; B65H 20/06

(52) U.S. Cl. CPC B65H 23/26; B65H 20/06; A61F 13/15804; A61F 13/15699; A61F 13/15577; A61F 2013/15821; A61F 2013/15967

(57) ABSTRACT

Heat is supplied to elements of a web processing system to resist adhesive contamination by buildup. A variety of heat sources can be used on a variety of web handling structures, such as rollers, idlers, conveyors, vacuum belts, or any other place where adhesive accumulation is undesired.

### RELATED APPLICATION

This application claims the benefit of co-pending U.S. Provisional Patent Application Ser. No. 62/080,613, filed 17 Nov. 2014.

### BACKGROUND OF THE INVENTION

This invention relates to processing webs of material for use in disposable products such as diapers and sanitary napkins. In particular, this invention relates to preventing buildup of adhesive materials on web processing equipment.

Generally, diapers comprise an absorbent insert or patch and a chassis, which, when the diaper is worn, supports the insert proximate a wearer's body. Additionally, diapers may include other various patches, such as tape tab patches, reusable fasteners and the like. The raw materials used in forming a representative insert are typically cellulose pulp, tissue paper, poly, nonwoven web, acquisition, and elastic, although application specific materials are sometimes utilized. Usually, most of the insert raw materials are provided in roll form, and unwound and applied in assembly line fashion.

In the creation of a diaper (and, oftentimes also in conjunction with feminine hygiene products), multiple roll-fed web processes are typically utilized.

To create an absorbent insert, the cellulose pulp is unwound from the provided raw material roll and pulverized by a pulp mill. Discrete pulp cores are formed by a core forming assembly and placed on a continuous tissue web. Optionally, super-absorbent powder or polymer (SAP) may be added to the pulp core. The tissue web is wrapped around the pulp core. The wrapped core is debulked by proceeding through a calendar unit, which at least partially compresses the core, thereby increasing its density and structural integrity. After debulking, the tissue-wrapped core is passed through a segregation or knife unit, where individual wrapped cores are cut. The cut cores are conveyed, at the proper pitch, or spacing, to a boundary compression unit.

While the insert cores are being formed, other insert components are being prepared to be presented to the boundary compression unit. For instance, the poly sheet is prepared to receive a cut core. Like the cellulose pulp, poly sheet material is usually provided in roll form. The poly sheet is fed through a splicer and accumulator, coated with an adhesive in a predetermined pattern, and then presented to the boundary compression unit. In addition to the poly sheet, which may form the bottom of the insert, a two-ply top sheet may also be formed in parallel to the core formation. Representative plies are an acquisition web material and a nonwoven web material, both of which are fed from material rolls, through a splicer and accumulator. The plies are coated with adhesive, adhered together, cut to size, and presented to the boundary compression unit. Therefore, at the boundary compression unit, three components are provided for assembly: the poly bottom sheet, the core, and the two-ply top sheet.

A representative boundary compress ion unit includes a die roller and a platen roller. When all three insert components are provided to the boundary compression unit, the nip of the rollers properly compresses the boundary of the insert. Thus, provided at the output of the boundary compression unit is a string of interconnected diaper inserts. The diaper inserts are then separated by an insert knife assembly and properly oriented. At this point, the completed insert is ready for placement on a diaper chassis.

A representative diaper chassis comprises nonwoven web material and support structure. The diaper support structure is generally elastic and may include leg elastic, waistband elastic and belly band elastic. The support structure is usually sandwiched between layers of the nonwoven web material, which is fed from material rolls, through splicers and accumulators. The chassis may also be provided with several patches, besides the absorbent insert. Representative patches include adhesive tape tabs and resealable closures.

The process utilizes two main carrier webs; a nonwoven web which forms an inner liner web, and an outer web that forms an outwardly facing layer in the finished diaper. In a representative chassis process, the nonwoven web is slit at a slitter station by rotary knives along three lines, thereby forming four webs. One of the lines is on approximately the center line of the web and the other two lines are parallel to and spaced a short distance from the centerline. The effect of such slicing is twofold; first, to separate the nonwoven web into two inner diaper liners. One liner will become the inside of the front of the diaper, and the second liner will become the inside of the back of that garment. Second, two separate, relatively narrow strips are formed that may be subsequently used to cover and entrap portions of the leg-hole elastics. The strips can be separated physically by an angularly disposed spreader roll and aligned laterally with their downstream target positions on the inner edges of the formed liners.

After the nonwoven web is sliced, an adhesive is applied, to the liners in a predetermined pattern in preparation to receive leg-hole elastic. The leg-hole elastic is applied to the liners and then covered with the narrow strips previously separated from the nonwoven web. Adhesive is applied to the outer web, which is then combined with the assembled inner webs having elastic thereon, thereby forming the diaper chassis. Next, after the elastic members have been sandwiched between the inner and outer webs, an adhesive is applied to the chassis. The chassis is now ready to receive an insert.

To assemble the final diaper product, the insert must be combined with the chassis. The placement of the insert onto the chassis occurs on a placement drum or at a patch applicator. The inserts are provided to the chassis on the placement drum at a desired pitch or spacing. The generally flat chassis/insert combination is then folded so that the inner webs face each other, and the combination is trimmed. A sealer bonds the webs at appropriate locations prior to individual diapers being cut from the folded and sealed webs.

Generally, disposable undergarments such as pants-type diapers are made up of two nonwoven layers of material with elastic strands of material placed between the two nonwoven layers of material thus creating an elastic web laminate. The layers of material are continuous sheets of material that are eventually cut into individual undergarment lengths. The elastic strands may be arranged and cut so that specific areas of the undergarment are free of elastic tension or forces. An absorbent pad, often contained within an insert or core is then also placed into the pants-type diaper product.

To insure the pants-type diaper retains a

proper shape and to hold all of the added layers of the diaper, reinforcing layers and backing materials are normally added to the continuous sheets of material, with the reinforcing layers corresponding to the cut elastic strands of each individual blank. Each of these layers needs to be adhesively joined, at some point in the manufacturing process to the elastic web laminate to form the completed undergarment.

Often, void spaces need to be created in the diaper, such as holes cut out of the main web for provided leg holes when the undergarment is ultimately formed. To create the void spaces, the web is ordinarily die cut, with the web severed between a die and an anvil. The portion of the web material that is removed is referred to as a “chip.” As the die wears throughout time, the severing of the chip from the web material becomes gradually a duller cut. This complicates the removal of the chip because the severing might not create a continuous cut out chip, with possibly some strands of the web material still coupling the chip with the web. It is desired to lengthen the amount of time and increase the number of chips that a single die can effectively be used for, to reduce the number of die change-outs.

Typically, the absorbent fibrous material is composed of cellulose wadding or cellulosic wood pulp material commonly referred to as “fluff”, although a mixture of natural and synthetic fibers is within the scope of the invention. An absorbent core composed of wood pulp fluff is typically formed by employing conventional air laying techniques.

These absorbent cores have had their total absorbency improved greatly by the addition of super absorbent material, or super absorbent polymer (SAP) to the commonly used absorbent fibrous materials.

The ability of these absorbent cores to manage the typical surges of liquid flow is heavily dependent on the proper distribution of super absorbent material within the absorbent fluff. When most super absorbent materials absorb aqueous fluids, they swell substantially, often, to double their dry dimensions or more at saturation. As these super absorbent materials absorb fluid and swell, they generally become a gelatinous mass.

There has been a trend in reducing the bulk of diapers, in attempts to make them more like underwear and to take up less shelf space in retailer's outlets. Generally, the thinner the diaper, the higher the concentration of super absorbent material required to produce the same level of absorbency. High levels of super absorbent material, however, tend to be more difficult to control and to maintain in position.

In conventional core forming processes, three-dimensional fluff receiving pockets rotate about a vacuum drum. The pockets typically include baffles and screens which permit airflow through the pockets. The fluff is applied to the fluff receiving pockets entrained in air applied to the pockets. The vacuum attracts the fluff to a screen-like mesh that forms the pockets. The fluff is retained by the pockets, and the amount of fluff builds up from the screen forming the pocket. However, some fluff passes through the screen of the pockets and into the vacuum stream that is drawing the fluff into the pocket. Thus, some fluff undesirably becomes entrained in the vacuum stream.

In conventional core forming process, it is desired to balance the amount of air urging the fluff towards the core forming pocket and the amount of vacuum used to retain the fluff within the pocket. Machine processes have become more complex as speeds of machines have increased, so air handling systems used in this process have greater demands placed on them. For instance, if the machine is running faster, pulp is required to be delivered to the core forming pocket quicker, necessitating a greater air flow to the pocket. To deliver more pulp to the pocket, more vacuum is required to retain the pulp within the pocket. One complication is in achieving optimum balance between air in to the pocket and vacuum applied to the back side of the pocket.

Imbalance between the amount of air supplying pulp to the core forming pocket and vacuum applied to the back of the pocket, holding the fluff in, causes puffs of fluff to escape forming chamber. Conventional core forming technology allows for limited adjustability to try and achieve the optimum balance between air in and vacuum. The largest air delivery is from fiberizing mill which supplies fluff and blows the fluff into the core forming chamber.

Another source of air into forming process is from the dust collection equipment, which returns collected fluff from the vacuum stream to the core forming drum. Beginning with fluff that passes through the core forming pocket, the vacuum stream leads the fluff within the vacuum stream to the dust collection unit. A filter within the dust collection unit captures this fluff, this fluff is removed from the filter, and recirculated into the core forming process. Typically, this vacuum stream is fed into a drum filter housing, such as described in U.S. Pat. No. 5,679,136, commercial embodiments of which are available from the Osprey Corporation, and which is incorporated herein by reference.

Sanitary napkins used in feminine hygiene are absorbent items worn by women to recover undesirable bodily discharges. These absorbent articles are typically comprised of an absorbent core sandwiched between layers of woven or non-woven materials.

In many adhesive applications used in hygienic disposable products, adhesive is applied on a porous web like a nonwoven and. the adhesive tends to pass through that web. Below the web there is usually a roller or conveying belt and the adhesive that passes through the web undesirably accumulates on these surfaces. Traditionally, undesirable adhesive accumulation has been addressed by making rollers with a release coating that attempts to minimize the ability of adhesives to grip to that surface. In some nip applications, chilled water cooling is used internally in rollers to try and reduce the adhesive accumulation.

### SUMMARY OF THE INVENTION

This invention relates to heated conveying surfaces and rollers which reduce undesirable adhesive accumulation.

Heating through a variety of techniques can be used, such as induction (which may be preferred for belts), infrared (IR) radiant heaters (which may be preferred to be focused to a narrow surface region), convective heat such as hot air or steam, external heaters, such as a hot roller against the target surface, and an electric cartridge heater placed inside of a hollow structure.

The heating techniques of the present invention can be used on a variety of web handling structures, such as rollers, idlers, conveyors, vacuum belts, or any other place where adhesive accumulation is undesired, or where it is desired to any surface that carries an adhesive coated nonwoven web.

In other areas of web processing units, a heated knife in a knife/anvil combination can eliminate complex and expensive equipment used to chill knives, the prior art method of reducing adhesive contamination on knife blades that come into contact with adhesive coated web material.

As an alternative, heat can be applied intermittently to reduce areas experience adhesive contamination. Heat could be added to a roller that was already experiencing contamination find the contamination is reduced as web material carries heated adhesive downstream.

### BRIEF DESCRIPTION OF THE DRAWINGS

FIG. 1 is a side view of an nonwoven material layer being applied with adhesive, and carried by a heated structure;

FIG. 2 is a side view of an exemplary heated structure;

FIG. 3 is a side view of an alternative embodiment of a heated structure;

FIG. 4 is a side view of a web conveyance system with a heat source heating a conveying surface to minimize adhesive accumulation on the conveying surface;

FIG. 5 is a heated roller upstream of a roller to illustrate the downstream benefit of heat to an upstream heated roller.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

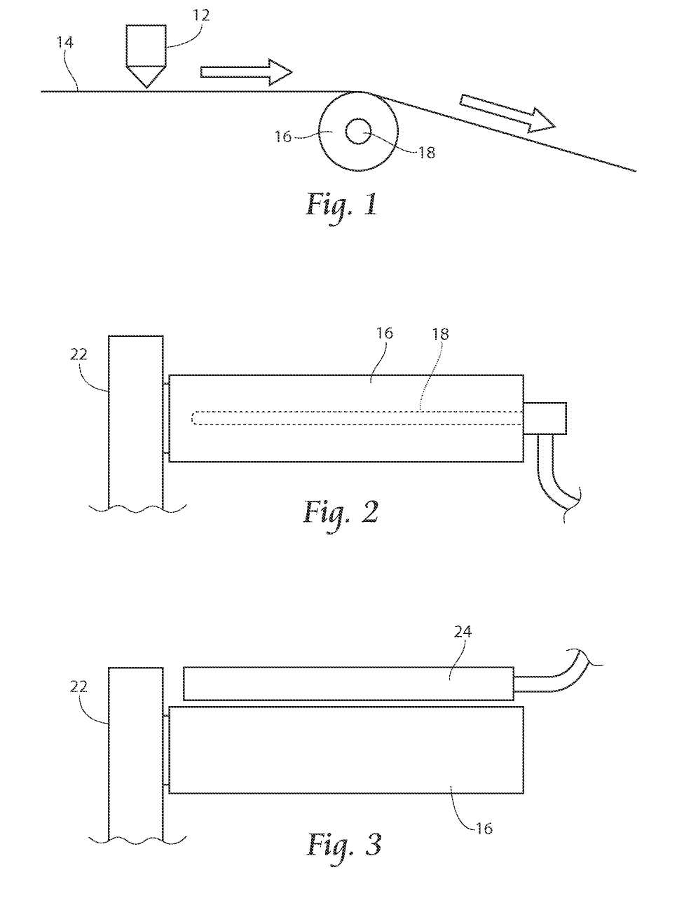

Referring now to FIG. 1, a heated, roller 16 carrying a nonwoven web 14 applied with adhesive by adhesive applicator 12 is shown.

Referring now to FIG. 2, a heater cartridge 18

powered by electrical leads is shown embedded in rotating cylinder or roller 16, which is carried by a mount 22. As shown in FIG. 3, instead of a heater cartridge 18, an induction unit 24 can be positioned proximally to the roller 16.

FIG. 3 is a side view of an alternative embodiment of a heated structure.

### Experiments

A cartridge heater was used to heat an approximately 1″ diameter roller in three conditions: 1) plasma coated; 2) a bare steel roller, and 3) a bare aluminum roller. The cartridge heater heated the roller from ambient temperature to approximately 300° F., which is approximately the melting point of nonwoven material used in the experiments. Elastic adhesive—(Bostik—H4356-C08) and construction adhesive (Henkel Easymelt 34-898B) were used as the adhesives which accumulation is to be prevented.

In an experiment conducted with a blue plasma coated roll, construction glue at a temperature of 300° F., and Easymelt 34-898B Additive, at a surface temperature of the roll at ambient room temperature and up to approximately a surface temperature of the roll of 150° F., adhesive buildup was noted, buildup decreasing as temperature approached 150° F. From approximately a surface temperature of the roll of 150° F. up to the melting point of the material layer carrying the adhesive, no buildup was observed. Similar results were achieved with elastic glue.

In an experiment conducted with a bare aluminum roll, construction glue at a temperature of 300° F., and Easymelt 34-898B Additive, at a surface temperature of the roll at ambient, room temperature and up to approximately a surface temperature of the roll of 150° F., adhesive buildup was noted, buildup decreasing as temperature approached 150° F., but buildup accumulating after longer material run durations. From approximately a surface temperature of the roll of 150° F. up to the melting point of the material layer carrying the adhesive, no buildup was observed. Similar results were achieved using elastic glue, Oreja Elastico H43560C08.

In an experiment conducted with a bare steel roll, construction glue at a temperature of 300° F., and Easymelt 34-898B Additive, at a surface temperature of the roll at ambient room temperature and up to approximately a surface temperature of the roll of 165° F., adhesive buildup was noted, buildup decreasing as temperature approached 165° F., but buildup accumulating after longer material run durations. From approximately a surface temperature of the roll of 165° F. up to the melting point of the material layer (which can be approximately 280° F.-300° F.) carrying the adhesive, no buildup was observed. Similar results were achieved using elastic glue, Oreja Elastico H43560C08.

From the experiments, it is concluded that adhesive accumulation is prevented by heating the roll beneath the nonwoven to a range of approximately about 150° F. to approximately about 300° F. which is the approximate melting point of the nonwoven. It is noted that a material layer capable of withstanding higher melting temperatures could also allow for higher heating in the roller if desired or necessary.

The heat may delay the set up of the adhesive by keeping the adhesive from developing its tack or keeping its strength low at the adhesive/roller interface so that the tendency of the adhesive is to travel with the nonwoven. Heating the roller surface may lower its surface energy enough to prevent wet out, which is thought to be a key first step for adhesion.

In a knife of heated knife in a knife/anvil combination, such as shown in U.S. Application 2015/0096177, incorporated herein by reference, the knife itself can be heated to reduce adhesive buildup. This is divergent than previous approaches on the subject, which concluded that chilled knives were the best approach to reduce adhesive buildup.

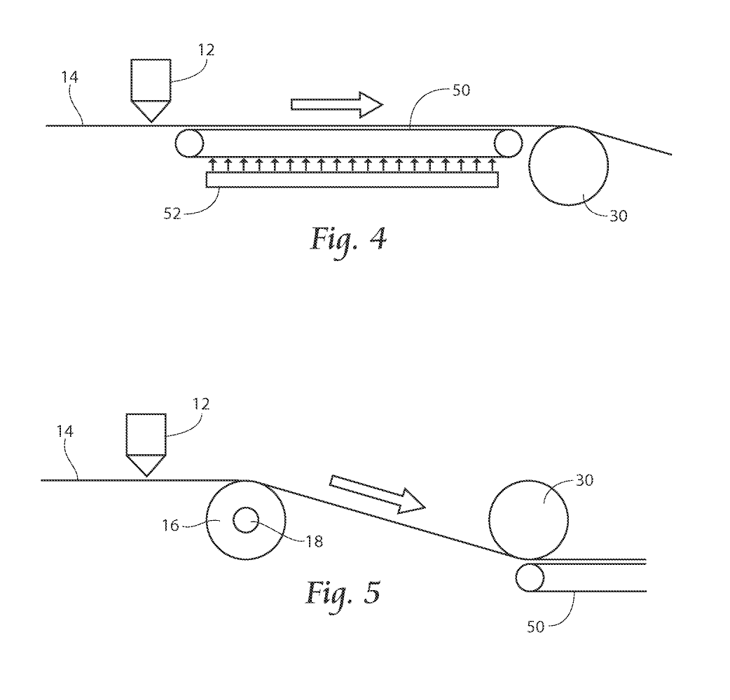

Referring now to FIG. 4, a side view of a web conveyance system 50 with a heat source 52 heating a conveying surface of web conveyance system 50 is shown. In one embodiment, the heat source 52 is a radiant heat source applying heat to conveyance system 50 to minimize adhesive accumulation on the conveying surface of conveyance system 50.

Referring now to FIG. 5 heated roller 16 is shown upstream of a roller 30 and conveyor 50. One unexpected result of heating roller 16, is that upstream heated roller 16 prevents adhesive from collecting downstream on rollers 30 and conveyor 50 when an upstream heating roller 16 is used. In complex processes or locations difficult to access for which adhesive contamination reduction is desired, a heated roller 16 may be installed just upstream a target location, rather than having to figure out how to bring heating into the complex process itself.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. An apparatus for processing a web of material, the apparatus comprising an operating body heated from an ambient temperature to an adhesive accumulation resistant temperature by a heat source acting upon said rotating body.

2. An apparatus according to claim 1, said operating body comprising a knife.

3. An apparatus according to claim 1, said operating body comprising a rotating body.

4. An apparatus according to claim 3, said heat source comprising a heating element internal to said rotating body.

5. An apparatus according to claim 3, said heat source comprising a heating element external to said rotating body.

6. An apparatus according to claim 3, said rotating body comprising a rotating drum.

7. An apparatus according to claim 6, said rotating drum comprised of a metallic material.

8. An apparatus according to claim 1, said heat source comprising a radiant heat source.

9. An apparatus according to claim 1, said operating body comprising a conveying belt.

10. An apparatus according to claim 1, said heat source comprising an induction heat source.

11. An apparatus according to claim 1, said adhesive accumulation resistant temperature comprising a range between approximately 150° F. to a meriting point of said web of material.

12. An apparatus according to claim 1, said adhesive accumulation resistant temperature comprising a range between 150° F. to 300° F.

13. An apparatus according to claim 1, said apparatus further comprising a source of adhesive to apply adhesive to said web of material, said source of adhesive upstream of said operating body in a machine direction.
